(19) United States
(12) Patent Application Publication
(10) Pub. No.: US 2025/0258142 A1
(43) Pub. Date: Aug. 14, 2025

(54) ULTRASONIC IMAGING SYSTEM AND PHOTOACOUSTIC IMAGE GENERATION METHOD

(71) Applicant: FUJIFILM Corporation, Tokyo (JP)

(72) Inventor: Hirozumi TAKESHIMA, Tokyo (JP)

(73) Assignee: FUJIFILM Corporation, Tokyo (JP)

(21) Appl. No.: 19/007,539

(22) Filed: Jan. 1, 2025

(30) Foreign Application Priority Data
Feb. 8, 2024 (JP) 2024-017806

(51) Int. Cl. G01N 29/24; G01N 29/06; G01N 29/30; G01N 29/44

(52) U.S. Cl. CPC G01N 29/2418; G01N 29/0672; G01N 29/30; G01N 29/4463

(57) ABSTRACT

An ultrasonic image system that generates a photoacoustic image is provided. A transducer array receives a photoacoustic wave from an optical absorption element in a state in which an insertion member is inserted into a living body. An analyzer calculates a synchronization deviation based on a reception signal sequence obtained in this manner. A plurality of correctors correct a plurality of reception signals based on the synchronization deviation. The plurality of corrected reception signals are subjected to phase addition. A photoacoustic image is generated based on the corrected reception information generated by the phase addition. The synchronization deviation may be calculated based on a pseudo-reception signal sequence.

### CROSS-REFERENCE TO RELATED APPLICATION

This application claims priority under 35 USC 119 from Japanese Patent Application No. 2024-017806, filed 8 Feb. 2024, the disclosure of which is incorporated by reference herein.

### BACKGROUND OF THE INVENTION

### 1. Field of the Invention

The present disclosure relates to an ultrasonic imaging system and a photoacoustic image generation method, and particularly, to a technique for displaying a position of an insertion member inserted into a living body.

### 2. Description of the Related Art

An ultrasonic imaging system is a system for performing treatment or examination on a living body which is a patient using an ultrasonic imaging apparatus. More specifically, in the ultrasonic imaging system, an image representing a position of an insertion member inserted into the living body is generated and displayed. The treatment or the like is performed on the living body with reference to such an image. From such a viewpoint, the ultrasonic imaging system is a system for supporting treatment or the like on a living body.

In the ultrasonic imaging system, an ultrasound diagnostic apparatus is usually used as the ultrasonic imaging apparatus. The insertion member to be inserted into the living body is, for example, a catheter to be inserted into a blood vessel. In general, a guide wire is inserted into a blood vessel before the insertion of the catheter. The guide wire is also the insertion member. An ultrasonic imaging method that images an insertion member inserted into a blood vessel using an ultrasound probe that comes into contact with a surface of a living body is also referred to as an extra-vascular ultrasound (EVUS).

An advanced ultrasonic imaging system that images an insertion member using a photoacoustic effect has been proposed. In such an ultrasonic imaging system, an optical absorption element is provided at a distal end of the insertion member. An optical pulse generated by an optical pulse generator is guided to the inside of the insertion member through an optical fiber, and the optical pulse is emitted to the optical absorption element. A photoacoustic wave is generated in the living body by the absorption of the optical pulse by the optical absorption element. The photoacoustic wave is received by an ultrasound probe that is in contact with a biological surface. An image (hereinafter, referred to as a photoacoustic image or a PA image) representing the position of the optical absorption element (that is, the sound source) is formed based on reception information obtained by the photoacoustic wave. For example, the photoacoustic image is composited with the ultrasound image (hereinafter, also referred to as a US image) generated by the ultrasonic transmission and reception. As a result, the generated composite image is displayed. Through an observation of the composite image, the position of the distal end of the insertion member can be clearly specified while observing the biological tissue.

In the above-described advanced ultrasonic imaging system, basically, it is necessary to synchronize an optical pulse period in the optical pulse generator with the reception period in the ultrasound probe. This is because, in a case in which the synchronization is not established, the reception beamforming is not appropriately executed, and a quality of the photoacoustic image is deteriorated. In addition, in order to accurately represent a depth position of the sound source in the photoacoustic image, it is necessary to match a generation timing of the optical pulse and a start timing of the reception period in the probe in addition to the matching of the optical pulse period and the reception period.

JP5819387B discloses the above-described advanced ultrasonic imaging system. JP5819387B does not disclose a technique of correcting a reception signal sequence. In the ultrasound diagnostic apparatus disclosed in JP2020-137876A, a Fourier transform (also referred to as Fourier phasing) and an inverse Fourier transform (also referred to as inverse Fourier phasing) are executed incrementally. The incremental data transformation is for sound velocity correction and is irrelevant to the photoacoustic image.

### SUMMARY OF THE INVENTION

An object of the present disclosure is, even in a case where there is a synchronization deviation between an optical pulse period and a reception period, to generate the same photoacoustic image as a photoacoustic image obtained in a case where there is no synchronization deviation. Alternatively, an object of the present disclosure is to display a sound source at a correct position in a photoacoustic image even in a case where there is a synchronization deviation between an optical pulse period and a reception period.

An ultrasonic imaging system according to the present disclosure comprises a light source configured to generate an optical pulse; an insertion member that is inserted into a living body and that includes an optical absorption element which converts the optical pulse into a photoacoustic wave; a probe including a plurality of transducers that receive the photoacoustic wave; an analyzer configured to calculate a synchronization deviation between an optical pulse period in the light source and a reception period in the probe based on a reception signal sequence consisting of a plurality of reception signals that are output in parallel from the plurality of transducers and a pseudo-reception signal sequence corresponding to the reception signal sequence; a processor configured to apply correction for eliminating a positional deviation caused by the synchronization deviation and phase addition for forming a reception beam to the reception signal sequence or the pseudo-reception signal sequence to output corrected reception information; and a generator configured to generate a photoacoustic image representing a position of the optical absorption element in the living body based on the corrected reception information.

A photoacoustic image generation method according to the present disclosure comprises receiving a photoacoustic wave with a plurality of transducers in a state in which an insertion member including an optical absorption element that converts an optical pulse into the photoacoustic wave is inserted into a living body; calculating a synchronization deviation between an optical pulse period and a reception period based on a reception signal sequence consisting of a plurality of reception signals that are output in parallel from the plurality of transducers and a pseudo-reception signal sequence corresponding to the reception signal sequence; applying correction for eliminating a positional deviation caused by the synchronization deviation and phase addition for forming a reception beam to the reception signal sequence or the pseudo-reception signal sequence to generate corrected reception information; and generating a photoacoustic image representing a position of the optical absorption element in the living body based on the corrected reception information.

According to the present disclosure, even in a case where there is a synchronization deviation between an optical pulse period and a reception period, the same photoacoustic image as a photoacoustic image obtained in a case where there is no synchronization deviation is generated. Alternatively, according to the present disclosure, even in a case where there is the synchronization deviation between the optical pulse period and the reception period, a sound source is displayed at a correct position in the photoacoustic image.

### BRIEF DESCRIPTION OF THE DRAWINGS

FIG. 1 is a block diagram showing an ultrasonic imaging system according to a first embodiment.

FIG. 2 is a diagram showing an example of a reception signal including a photoacoustic wave signal.

FIG. 3 is a diagram showing correction of a reception signal sequence before phase addition.

FIG. 4 is a diagram showing a spatial relationship between a sound source and a transducer array.

FIG. 5 is a diagram showing a relationship between a synchronization deviation, a real propagation time, and an apparent propagation time.

FIG. 6 is a diagram showing an example of a synchronization deviation calculation method.

FIG. 7 is a block diagram showing a first modification example.

FIG. 8 is a block diagram showing a second modification example.

FIG. 9 is a block diagram showing an ultrasonic imaging system according to a second embodiment.

### DESCRIPTION OF THE PREFERRED EMBODIMENTS

Hereinafter, the embodiment will be described with reference to the drawings.

### (1) Summary of Embodiment

The ultrasonic imaging system according to the embodiment includes a light source, an insertion member, a probe, an analyzer, a processor, and a generator. The light source generates an optical pulse. The insertion member is inserted into a living body. The insertion member includes an optical absorption element that converts an optical pulse into a photoacoustic wave. The probe comprises a plurality of transducers that receive the photoacoustic wave. The analyzer calculates a synchronization deviation between an optical pulse period in the light source and a reception period in the probe based on a reception signal sequence consisting of a plurality of reception signals output in parallel from the plurality of transducers or a pseudo-reception signal sequence corresponding to the reception signal sequence. The processor applies correction for eliminating a positional deviation caused by the synchronization deviation and phase addition for forming a reception beam to the reception signal sequence or the pseudo-reception signal sequence to output corrected reception information. The generator generates a photoacoustic image representing a position of the optical absorption element in the living body based on the corrected reception information.

According to the above configuration, correction for eliminating the positional deviation caused by the synchronization deviation is applied to the reception signal sequence or the pseudo-reception signal sequence. Therefore, even in a case where there is the synchronization deviation between the optical pulse period and the reception period, the influence of the synchronization deviation does not appear on the photoacoustic image, or the influence of the synchronization deviation appearing on the photoacoustic image is reduced. Therefore, the sound source is displayed at a correct position or a position close to the correct position in the photoacoustic image. In other words, according to the above-described configuration, since it is not necessary to strictly control the optical pulse period and/or the reception period, the configuration of the ultrasonic imaging system can be simplified. The above configuration may be made to function during a temporary period until synchronization is established or during a temporary period in which synchronization is interrupted.

From the viewpoint of calculating the synchronization deviation, the pseudo-reception signal sequence can be regarded as the same reception signal sequence as the reception signal sequence output from the plurality of transducers. For example, the pseudo-reception signal sequence may be generated by restoration processing on reception frame data or display frame data. The corrected reception information is reception frame data or display frame data to which correction for eliminating the positional deviation caused by the synchronization deviation is applied.

In the embodiment, the synchronization deviation is a time difference between a generation timing of the optical pulse and a start timing of the reception period in the probe. The start timing of the reception period is a reference timing in the reception beamforming. In a case where the generation timing of the photoacoustic wave matches the reference timing, a depth of the sound source is correctly specified. In other words, the depth of the sound source can be correctly expressed by eliminating the positional deviation due to the time difference in the reception signal sequence or the pseudo-reception signal sequence.

In the embodiment, the processor includes a correction unit and a phase addition unit. The correction unit shifts a photoacoustic wave signal sequence included in the reception signal sequence or the pseudo-reception signal sequence in a depth direction based on the synchronization deviation. The phase addition unit applies phase addition to the corrected reception signal sequence or the corrected pseudo-reception signal sequence output from the correction unit to generate corrected reception information.

The positional deviation of the photoacoustic wave signal sequence occurs on a time axis due to the synchronization deviation. The above-described configuration shifts the photoacoustic wave signal sequence along the time axis so that the positional deviation is eliminated. With the phase addition after shifting, an advantage that is not necessary to set a special phase addition condition can be obtained.

In the embodiment, the processor applies the correction and the phase addition to the reception signal sequence or the pseudo-reception signal sequence at the same time. According to this configuration, the configuration of the reception unit can be simplified. For example, by controlling the readout timing of each reception signal from each memory, it is possible to realize the simultaneous application of the correction and the phase addition to the reception signal sequence.

The ultrasonic imaging system according to the embodiment includes a restoration processor that applies restoration processing to the reception information generated from the reception signal sequence to generate a pseudo-reception signal sequence. The analyzer calculates the synchronization deviation based on the pseudo-reception signal sequence. According to this configuration, the positional deviation can be eliminated without changing the configuration of the reception unit. The restoration processing is processing of restoring the reception signal sequence before the phase addition from the reception information after the phase addition. The restored reception signal sequence is the pseudo-reception signal sequence.

In the embodiment, the analyzer calculates the synchronization deviation based on the photoacoustic wave signal sequence included in the reception signal sequence or the pseudo-reception signal sequence. In the embodiment, the photoacoustic wave signal sequence is composed of a plurality of photoacoustic wave signals corresponding to a plurality of transducers. The analyzer calculates the synchronization deviation based on a plurality of propagation times corresponding to the plurality of photoacoustic wave signals.

A plurality of photoacoustic wave signals constituting the photoacoustic wave signal sequence are arranged in a form in which a magnitude of the synchronization deviation is reflected in a two-dimensional space defined by a time direction (depth direction) and an electronic scanning direction. That is, the magnitude of the synchronization deviation is reflected in the plurality of propagation times corresponding to the plurality of photoacoustic wave signals. Using this, the synchronization deviation is calculated from the plurality of propagation times.

The photoacoustic image generation method according to the embodiment includes a reception step, an analysis step, a processing step, and a generation step. In the reception step, the photoacoustic waves are received by the plurality of transducers in a state in which the insertion member having the optical absorption element that converts the optical pulse into the photoacoustic wave is inserted into the living body. In the analysis step, the synchronization deviation between the optical pulse period and the reception period is calculated based on the reception signal sequence consisting of the plurality of reception signals output in parallel from the plurality of transducers or a pseudo-reception signal sequence corresponding to the reception signal sequence. In the processing step, the correction for eliminating the positional deviation caused by the synchronization deviation and phase addition for forming the reception beam are applied to the reception signal sequence or the pseudo-reception signal sequence to generate corrected reception information. In the generation step, a photoacoustic image representing a position of the optical absorption element in the living body is generated based on the corrected reception information.

### (2) Details of Embodiment

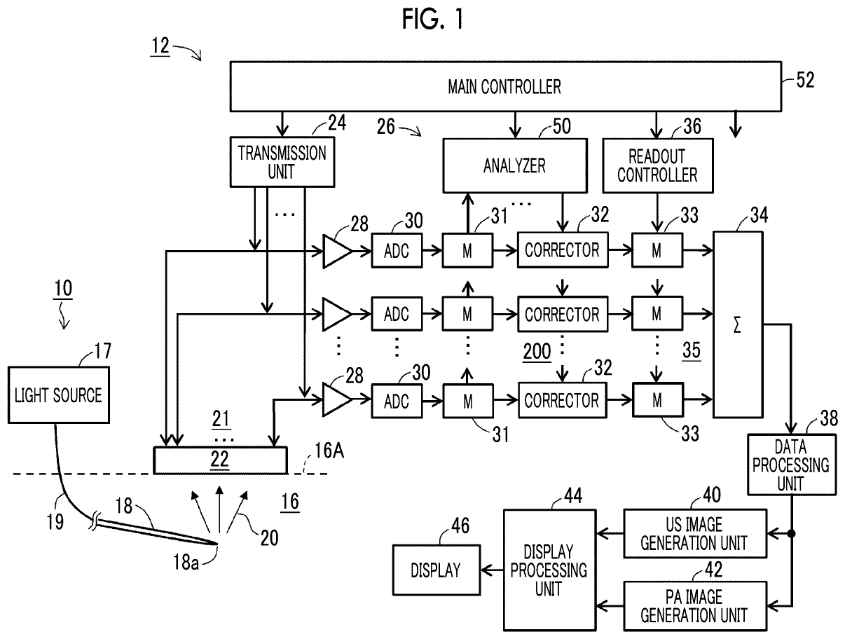

FIG. 1 shows an ultrasonic imaging system according to a first embodiment. The illustrated ultrasonic imaging system is a medical system installed in a medical institution such as a hospital, and specifically, is a system used in a case of sending a distal end of an insertion member to a diseased part while observing a photoacoustic image (PA image) representing a position of the distal end of the insertion member. The ultrasonic imaging system may be used for other applications.

The ultrasonic imaging system includes an optical pulse generation apparatus 10, an ultrasound diagnostic apparatus 12, and an insertion member 18. The ultrasound diagnostic apparatus 12 functions as an ultrasonic imaging apparatus. In the illustrated configuration example, an apparatus for establishing synchronization between the optical pulse generation apparatus 10 and the ultrasound diagnostic apparatus 12 is not provided.

The optical pulse generation apparatus 10 includes a light source 17. The light source 17 is a laser that generates a pulse sequence of laser light. A proximal end of the optical fiber 19 is connected to the light source 17. A distal end of the optical fiber 19 is located in a distal end part of the insertion member 18.

The insertion member 18 is, for example, a catheter inserted into a blood vessel in the living body 16. Other examples of the insertion member 18 include a guide wire, a puncture needle, or the like. An optical absorption element 18a made of an optical absorption material is provided in the distal end part of the insertion member 18. In a case where the optical pulse is applied to the optical absorption element 18a, the optical absorption element 18a absorbs the optical pulse. At that time, the photoacoustic wave 20 is generated by the photoacoustic effect. The photoacoustic wave 20 propagates through the living body 16 as a pulsed wave. The optical absorption element 18a functions as a sound source in the living body 16. By imaging the sound source, the position of the distal end of the insertion member 18 can be specified in real time.

Next, the ultrasound diagnostic apparatus 12 will be described. The ultrasound diagnostic apparatus 12 has a B-mode in which a B-mode tomographic image (US image) is displayed, and a PA-mode in which a photoacoustic image (PA image) is displayed.

The ultrasound probe 21 is in contact with a surface 16A of the living body 16. The ultrasound probe 21 is usually held by an examiner. The ultrasound probe 21 may be held by a fixation instrument, a robot arm, or the like. A transducer array 22 composed of the plurality of transducers is provided in the ultrasound probe 21.

In a case where the B-mode is executed, the ultrasound wave transmitted from the transducer array 22 is radiated into the living body 16, and the reflected wave from the living body 16 is received by the transducer array 22. More specifically, a transmission beam and a reception beam are formed and are electronically scanned.

In a case where the PA-mode is executed, the transducer array 22 does not perform a transmission operation and only performs a reception operation. That is, only the reception beam is formed and is subjected to electronic scanning. More specifically, the photoacoustic wave 20 generated in the living body 16 is received by the transducer array 22. A plurality of reception periods are set on the time axis in accordance with the reception period. Each reception period is a period for receiving or detecting a photoacoustic wave, and corresponds to a reception beam forming period.

It should be noted that a plurality of reception beams may be simultaneously and parallelly formed in each reception period. The reception signal sequence obtained from the entire transducer array 22 may be used as the reception information for the synchronization deviation analysis, and a part of the reception signal sequence may be used as the reception information for the reception beam formation. In general, a transmission and reception operation according to the B-mode and a reception operation according to the PA-mode are alternately executed.

The transmission unit 24 is a transmission beam former. That is, the transmission unit 24 outputs a plurality of transmission signals in parallel to the plurality of transducers at the time of transmission. The transmission unit 24 is configured by, for example, a processor. The processor may be configured by a programmable device, an electronic circuit, or the like.

The reception unit 26 is a reception beam former. Specifically, the reception unit 26 processes a plurality of reception signals output in parallel from the plurality of transducers at the time of reception. The reception unit 26 is configured by a processor. The processor functions as an analyzer 50, a correction unit 200, and a phase addition unit 35, which will be described below. The analyzer 50, the correction unit 200, and the phase addition unit 35 may be configured by a processor, respectively.

Specifically, the reception unit 26 includes a plurality of amplifiers 28, a plurality of ADCs 30, a plurality of memories (first memories) 31, a plurality of correctors 32, a plurality of memories (second memories) 33, an adder 34, a readout controller 36, an analyzer 50, and the like. The plurality of memories 33, the adder 34, and the readout controller 36 constitute a phase addition unit 35.

The plurality of amplifiers 28 amplify the plurality of input reception signals. The plurality of ADCs 30 convert a plurality of amplified reception signals (analog signals) into a plurality of digital signals. A plurality of reception signals output from the plurality of ADCs 30 are temporarily stored in the plurality of memories 31. The plurality of correctors 32 apply correction processing to the plurality of reception signals output from the plurality of memories 31. The correction processing is processing of eliminating the positional deviation caused by the synchronization deviation. This will be described in detail below. The plurality of reception signals output from the plurality of correctors 32 are temporarily stored in the plurality of memories 33.

The plurality of reception signals are read out from the plurality of memories 33 by the readout control of the readout controller 36. The plurality of read-out reception signals are added by the adder 34. As a result, reception beam data is generated. In other words, the readout controller 36 dynamically gives a delay time to each reception signal such that the reception beam data is generated, that is, the reception focus is formed at each depth on the sound source.

The reception unit 26 outputs the reception beam data generated by the phase addition. A plurality of pieces of reception beam data arranged in an electronic scanning direction are generated by one electronic scanning of a reception beam. The reception frame data corresponding to the beam scanning plane is composed of the plurality of pieces of reception beam data. Each reception beam data is composed of a plurality of pieces of echo data arranged in a depth direction.

The reception unit 26 according to the embodiment includes a plurality of memories 31 and a plurality of correctors 32 in order to eliminate the positional deviation caused by the synchronization deviation, and further includes an analyzer 50. The configurations do not function in a case where the B-mode is executed, and function in a case where the PA-mode is executed. The correction unit 200 is configured by the plurality of correctors 32.

The analyzer 50 calculates a synchronization deviation (synchronization deviation amount) based on a plurality of reception signals, that is, reception signal sequences before phase addition, stored in the plurality of memories 31. The synchronization deviation can also be referred to as a phase deviation. The analyzer 50 can be configured by, for example, a processor.

In general, in a state where there is no synchronization deviation, a temporal relationship between the optical pulse period and the reception period is constant. In a state where the synchronization deviation occurs, the temporal relationship between the optical pulse period and the reception period changes from moment to moment. In the embodiment, the synchronization deviation is calculated in order to display the sound source at a correct depth on the displayed image. Specifically, in a state where the temporal relationship between the optical pulse period and the reception period is constant, a time difference between a generation timing of each optical pulse and a start timing of each reception period is calculated as a synchronization deviation. The synchronization deviation appears as a positional deviation in a depth direction in a two-dimensional space defined by the depth direction and the electronic scanning direction. The calculation method of the synchronization deviation will be described in detail below. In the process in which the temporal relationship between the optical pulse period and the reception period changes, the time difference may be repeatedly calculated as the synchronization deviation.

A plurality of reception signals output from the plurality of memories 31 are input to the plurality of correctors 32. Each corrector 32 corrects each reception signal based on the synchronization deviation calculated by the analyzer 50. Specifically, the position of the photoacoustic wave included in each reception signal is shifted in the time axis direction. This shift corresponds to the elimination of the synchronization deviation, that is, the elimination of the positional deviation. The plurality of corrected reception signals are transmitted to the phase addition unit 35. Therefore, with the configuration shown in FIG. 1, the reception signal sequence representing a correct sound source position is obtained.

In a case where the photoacoustic wave cannot be observed due to the fact that the photoacoustic wave has reached the transducer array 22 at a time other than the reception period, at least one of the optical pulse period or the reception period may be automatically or changed on a trial basis by a user. For example, the analyzer 50 may determine whether or not the photoacoustic wave component is included in the reception signal sequences stored in the plurality of memories 31. In a case where it is determined that the photoacoustic wave component is not included, at least one of the optical pulse period or the reception period may be changed.

The data processing unit 38 is configured by a processor that processes each reception beam data. The data processing unit 38 includes an envelope detection circuit, a filter circuit, a logarithmic conversion circuit, and the like. In a case of generating an ultrasound image (US image), each reception beam data output from the data processing unit 38 is sent to the US image generation unit 40. In a case of generating a photoacoustic image (PA image), each reception beam data output from the data processing unit 38 is sent to the PA image generation unit 42.

The US image generation unit 40 functions in a case where the B-mode is executed. The US image generation unit 40 is a module that has a digital scan converter (DSC) and generates display frame data from the reception frame data. Specifically, the US image generation unit 40 generates a tomographic image (B-mode tomographic image) representing a tissue structure as the US image. The DSC has a coordinate transformation function, a pixel interpolation function, and the like.

The PA image generation unit 42 functions in a case where the PA-mode is executed. The PA image generation unit 42 is a module that has the DSC and the sound source specifying unit and generates the display frame data from the reception frame data. The sound source specifying unit specifies a position of the sound source in the living body by detecting or extracting a sound source signal included in the display frame data generated by the DSC. The PA image includes a marker representing a position of the sound source in the beam scanning plane. The marker is, for example, a point having high brightness or a predetermined color. Since the positional deviation caused by the synchronization deviation has already been eliminated by the correction processing in the reception unit 26, the marker is displayed at the correct position in the PA image.

The display processing unit 44 generates a composite image by superimposing the PA image on the US image (tomographic image). The composite image is displayed on the display 46. The display 46 is configured by an organic EL display device, a liquid crystal display device, or the like.

The US image generation unit 40, the PA image generation unit 42, and the display processing unit 44 may be configured by a processor, respectively. The CPU described below may function as the US image generation unit 40, the PA image generation unit 42, and the display processing unit 44.

A main controller 52 controls the operation of each element constituting the ultrasound diagnostic apparatus 12. The main controller 52 is configured with, for example, a CPU that executes a program. The main controller 52 may function as all or a part of an element group shown in FIG. 1.

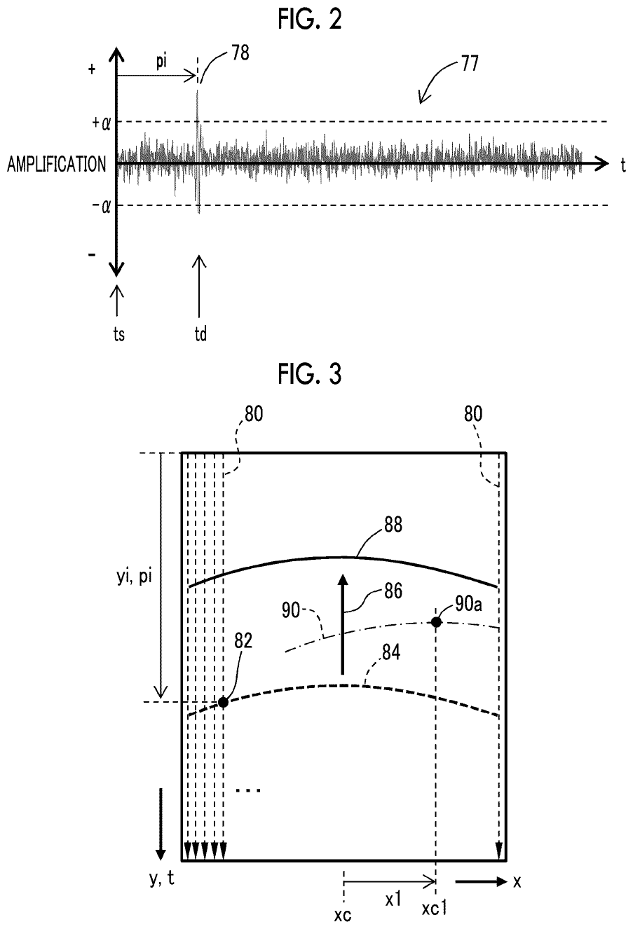

FIG. 2 shows a reception signal before correction. The reception signal 77 includes a photoacoustic wave signal 78 caused by the reception of the photoacoustic wave. The photoacoustic wave signal 78 has a peak-like or pulse-like form having a large amplitude. For example, the photoacoustic wave signal 78 can be detected or extracted by threshold value processing. In that case, a portion exceeding the threshold value α is specified as the photoacoustic wave signal 78. The threshold value α may be set according to the magnitude of the noise included in the reception signal. For example, in a case in which a standard deviation of the reception signal is represented by σ, the threshold value α may be set in accordance with α=6σ. Prior to the threshold value processing, filter processing, envelope detection, or the like may be applied to the reception signal 77. The photoacoustic wave signal 78 may be specified by detection of a maximum value, comparison with a reference waveform (for example, cross-correlation calculation), and the like.

The detection timing td is specified by the detection of the photoacoustic wave signal 78. The period pi from the reception period start timing ts to the detection timing td includes a time corresponding to the propagation time of the photoacoustic wave and the synchronization deviation. From such a viewpoint, in the following, the period pi will be referred to as an apparent propagation time.

FIG. 3 shows an example of a reception signal sequence referred to by the analyzer. The reception signal sequence is composed of a plurality of reception signals 80 corresponding to the plurality of transducers constituting the transducer array. In FIG. 3, the x direction is the transducer array direction (electronic scanning direction), and the y direction is the depth direction. The y direction corresponds to a time axis.

The reception signal sequence includes a photoacoustic wave signal sequence 84. In the shown example, an x coordinate (xc) of a center of the transducer array matches an x coordinate of the sound source, and an x coordinate of an apex of the photoacoustic wave signal sequence 84 matches the x coordinate (xc) of the center of the transducer array.

The photoacoustic wave signal sequence 84 is composed of a plurality of photoacoustic wave signals. Focusing on a specific photoacoustic wave signal 82 received by the i-th transducer, the apparent propagation time pi is specified based on the detection timing. The specific photoacoustic wave signal 82 is generated at a point at a depth yi.

The photoacoustic wave signal sequence 84 has a parabolic form. The form of the photoacoustic wave signal sequence 84 depends on the depth of the sound source. The form thereof is constant regardless of the magnitude of the synchronization deviation. A position where the photoacoustic wave signal sequence 84 is generated varies depending on a spatial relationship between the transducer array and the sound source, and varies depending on the synchronization deviation. For example, in a case in which a position of the sound source is shifted in the x direction from a center position xc of the transducer array, the photoacoustic wave signal sequence 90 is generated. An x coordinate (xc1) of the apex 90a corresponds to the x coordinate of the sound source. Even in that case, the form of the photoacoustic wave signal sequence 90 is the same as the form of the photoacoustic wave signal sequence 84.

The synchronization deviation is calculated based on the photoacoustic wave signal sequence 84. The synchronization deviation is a deviation of a generation timing of the photoacoustic wave with respect to a start timing of the reception period on a time axis. The synchronization deviation appears as a positional deviation in the depth direction of the photoacoustic wave signal sequence 84 in the two-dimensional coordinate system shown in FIG. 3.

As will be described later, the plurality of reception signals 80 are corrected based on the synchronization deviation. Specifically, the position of the photoacoustic wave signal sequence 84 is changed in the depth direction. In this case, on the two-dimensional coordinate system shown in FIG. 3, processing of moving the photoacoustic wave signal sequence 84 in parallel with the depth direction is applied to the photoacoustic wave signal sequence 84 (see reference numeral 86). Reference numeral 88 indicates a photoacoustic wave signal sequence in which the positional deviation caused by the synchronization deviation is eliminated.

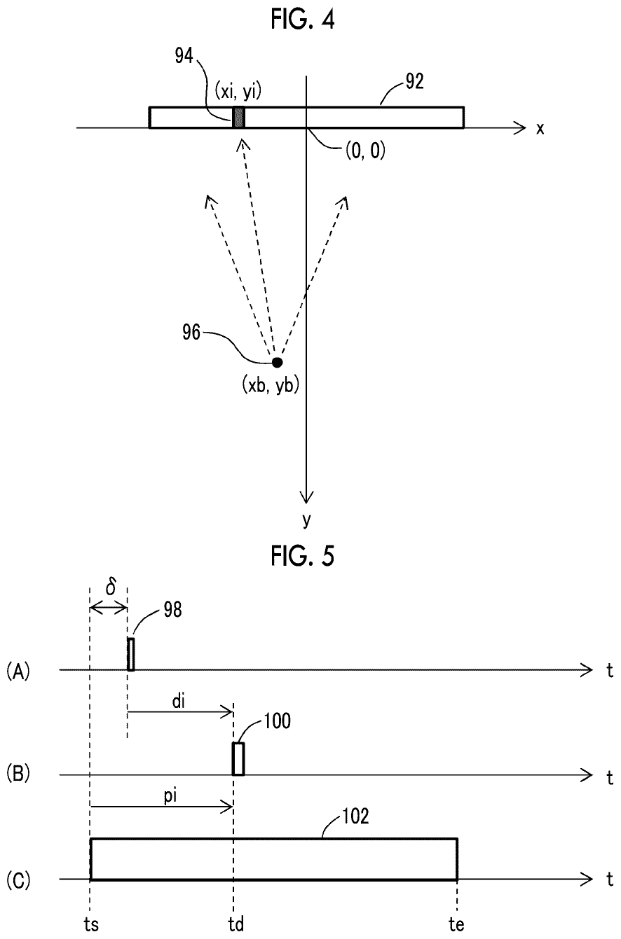

Hereinafter, a calculation method of the synchronization deviation will be described. FIG. 4 shows a spatial relationship between the transducer array 92 and the sound source 96. The x direction is the transducer array direction, and the y direction is the depth direction. The center of the transducer array 92 is the origin (0, 0), and the position of the i-th transducer 94 is represented by (xi, yi). However, yi=0. The position of the sound source 96 is represented by (xb, yb). The propagation time di of the photoacoustic wave from the sound source 96 to the i-th transducer is calculated by the following Equation (1). c in the Equation (1) is a sound velocity of an ultrasound wave in a biological tissue.

$${d}_{i} = \frac{\sqrt{{( {x}_{i} - {x}_{b} )}^{2} + {y}_{b}^{2}}}{c} \qquad ( 1 )$$

The apparent propagation time pi is a time obtained by adding the synchronization deviation δ and the propagation time (real propagation time) di. That is, the apparent propagation time pi is represented by the following Equation (2).

$${p}_{i} = δ + \frac{\sqrt{{( {x}_{i} - {x}_{b} )}^{2} + {y}_{b}^{2}}}{c} \qquad ( 2 )$$

A relationship between the apparent propagation time pi, the synchronization deviation δ, and the propagation time di, which is represented in the above Equation (2), is shown in FIG. 5. The reception period 102 is a period from the start timing ts to the end timing te. A time between the start timing ts and the generation timing of the optical pulse 98 is the synchronization deviation δ. A propagation time from the generation timing of the optical pulse 98 (generation timing of the photoacoustic wave) to reception of the photoacoustic wave by the i-th transducer is di. Reference numeral 100 indicates the photoacoustic wave signal generated by the reception of the photoacoustic wave.

In a case in which the photoacoustic wave reaches the n pieces of transducers, the n pieces of detection timings td corresponding to the n pieces of transducers are specified. The n pieces of apparent propagation times pi are specified based on the n pieces of detection timings td. The n pieces of data pairs (xi, pi) are defined by the positions xi of the n pieces of transducers and the n pieces of apparent propagation times pi (where, i=1, . . . , n).

The unknown parameters δ, xb, and yb can be specified by substituting the n pieces of data pairs (xi, pi) into Equation (2) which is a mathematical model. In this case, a solution search method such as a least squares method is used. In this method, in addition to the synchronization deviation δ, the coordinates (xb, yb) of the sound source are also specified.

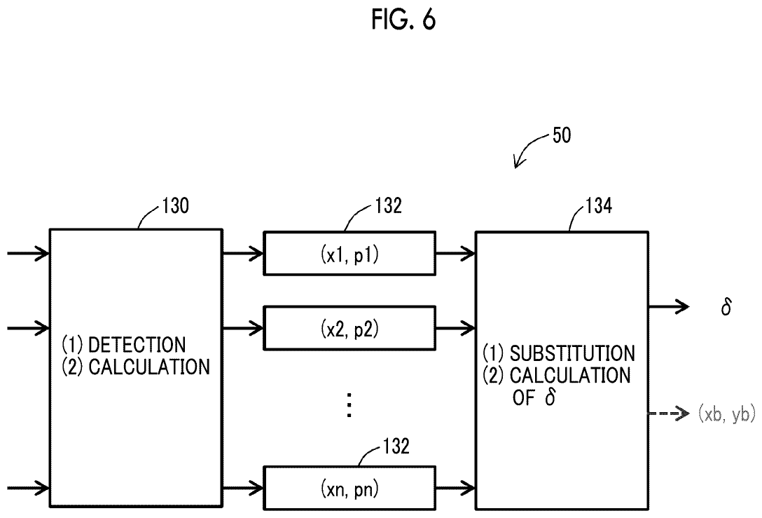

The process described above is schematically shown in FIG. 6. In the analyzer 50, in the block 130, the n pieces of photoacoustic wave signals included in the n pieces of reception signals before phase addition are detected, and the n pieces of apparent propagation times pi from the reception period start timing to the n pieces of detection timing are calculated. The n pieces of data pairs (xi, pi) 132 are defined by the coordinates xi of the n pieces of vibration elements in the x direction and the n pieces of apparent propagation times pi. In the block 134, the calculation of the synchronization deviation δ, which is the unknown parameter, is performed by substituting the n pieces of data pairs (xi, pi) 122 into Equation (2). The coordinates (xb, yb) of the sound source are also calculated secondarily. Only a plurality of data pairs (xi, pi) 122 satisfying certain conditions among the n pieces of data pairs (xi, pi) 122 may be substituted into Equation (2).

It should be noted that, in a case of focusing on |xi−xb| in Equation (2), in many cases, |xi−xb|<<yb is established. Therefore, the following Equation (3) is established based on Equation (2).

$${p}_{i} = δ + \frac{{y}_{b}}{c} \sqrt{1 + {( \frac{{x}_{i} - {x}_{b}}{{y}_{b}} )}^{2}} ≃ δ + \frac{{y}_{b}}{c} + \frac{1}{2 {y}_{b} c} {( {x}_{i} - {x}_{b} )}^{2} \qquad ( 3 )$$

Equation (3) indicates that the plurality of photoacoustic wave signals draw a parabola in the xy coordinate system. The synchronization deviation δ may be specified by using Equation (3). The photoacoustic image may be generated by using the coordinates (xb, yb) of the specified sound source, or the operation of the ultrasonic imaging system may be controlled based on the coordinates (xb, yb) of the specified sound source.

Incidentally, in the correction processing of eliminating the positional deviation caused by the synchronization deviation, the smoothed synchronization deviation may be referred to instead of the synchronization deviation calculated at each time point. In that case, for example, the smoothed synchronization deviation may be calculated according to Equation (4).

$$δ = \frac{1}{N} {\sum }_{0}^{N - 1} {δ}_{j} \qquad ( 4 )$$

In Equation (4), δⱼ represents the j-th synchronization deviation. Equation (4) corresponds to the moving average processing, and the most recent N pieces of the synchronization deviations are averaged by Equation (4).

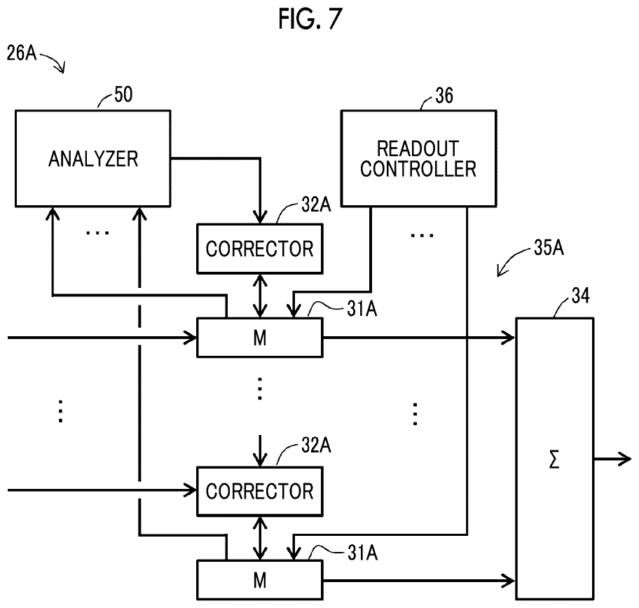

FIG. 7 shows a first modification example of the first embodiment. FIG. 7 shows a part of the reception unit 26A. A plurality of reception signals are stored in a plurality of memories 31A. The analyzer 50 analyzes the plurality of stored reception signals to specify the synchronization deviation.

A plurality of correctors 32A are connected to the plurality of memories 31A. Each corrector 32A reads out the reception signal from the corresponding memory 31A, corrects the reception signal based on the synchronization deviation, and writes the corrected reception signal in the memory 31A. In each memory 31A, the corrected reception signal is overwritten on the reception signal before correction.

Under the control of the readout controller 36, a plurality of corrected reception signals are read out from the plurality of memories 31A, and the plurality of read-out reception signals are added by the adder 34. As a result, the corrected reception information is obtained. A phase adder 35A is configured by the plurality of memories 31A, the adder 34, and the readout controller 36. According to the first modification example, the number of memories in the reception unit 26A can be reduced.

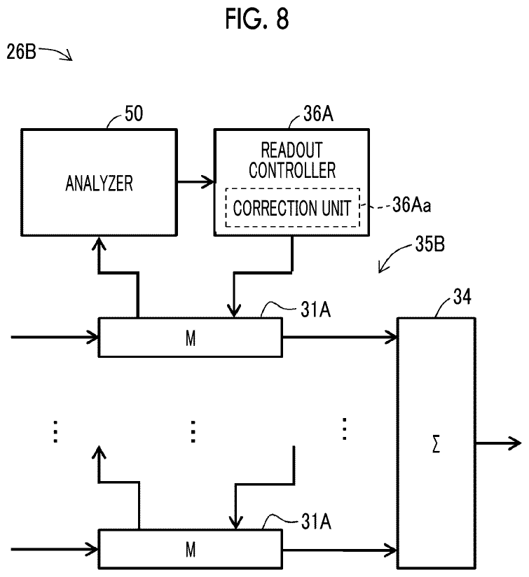

FIG. 8 shows a second modification example of the first embodiment. FIG. 8 shows a part of the reception unit 26B. A plurality of reception signals are stored in a plurality of memories 31A. The analyzer 50 analyzes the plurality of stored reception signals to specify the synchronization deviation.

The readout controller 36A has a delay control function for phase addition and a delay control function for positional deviation correction. In FIG. 8, the latter delay control function is expressed as the correction unit 36Aa. An actual delay amount determined from a first delay amount for phase addition and a second delay amount for positional deviation correction is applied to each reception signal. Whether to add or subtract the second delay amount from the first delay amount is determined according to the polarity (positive/negative) of the synchronization deviation.

The plurality of reception signals are read out from the plurality of memories 31A under the control of the readout controller 36A. The reception signals are added by the adder 34. As a result, the corrected reception information is obtained. The plurality of memories 31A, the adder 34, and the readout controller 36A constitute a phase adder 35B. According to the second modification example, the configuration of the reception unit 26B is simplified. Instead of the phase addition to the plurality of reception signals, the phase addition to a plurality of photoacoustic wave signals extracted from the plurality of reception signals may be performed.

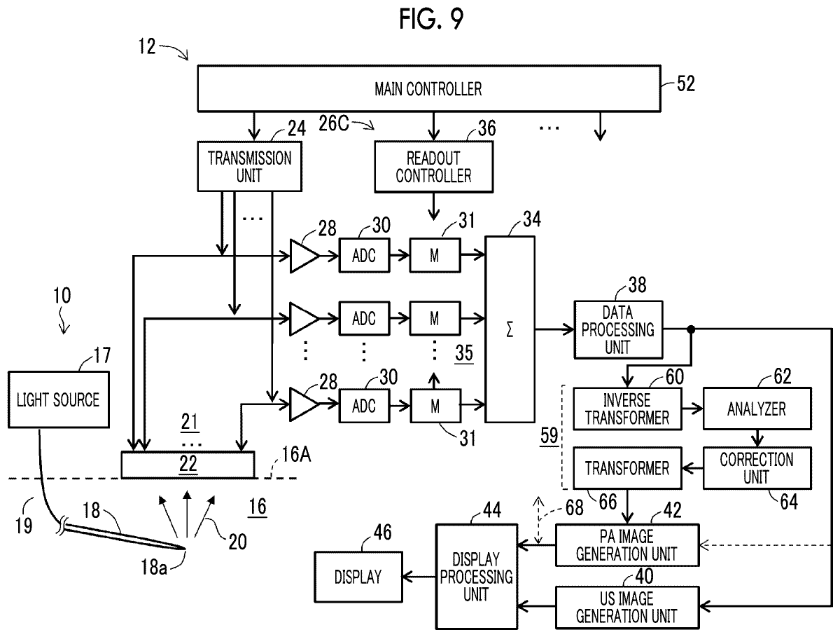

FIG. 9 shows an ultrasonic imaging system according to a second embodiment. In FIG. 9, the same elements as the elements shown in FIG. 1 are designated by the same reference numerals, and the description thereof will be omitted.

In the second embodiment, in a case where the B-mode is executed, the reception information (the reception frame data after the phase addition) output from the data processing unit 38 is sent to the US image generation unit 40. In a case where the PA-mode is executed, the reception information (the reception frame data after the phase addition) output from the data processing unit 38 is sent to the processor 59. The reception unit 26C has a general configuration.

The processor 59 includes an inverse transformer 60, an analyzer 62, a correction unit 64, and a transformer 66. The inverse transformer 60 is a restoration processor or a restorer that applies restoration processing to the reception information. The restoration processing is processing of generating the reception signal sequence before the phase addition from the reception information after the phase addition. From this viewpoint, the restoration processing is an inverse transformation (inverse phase addition) corresponding to the phase addition. The restoration processing may be an inverse Fourier transform (inverse Fourier phasing). In this way, the pseudo-reception signal sequence corresponding to the reception signal sequence before the phase addition is generated by the inverse transformer 60.

In a case in which the pseudo-reception signal sequence is mapped onto the xy coordinate space, the same photoacoustic wave signal sequence as the photoacoustic wave signal sequence 84 shown in FIG. 3 is generated. In FIG. 9, the analyzer 62 calculates the synchronization deviation based on the photoacoustic wave signal sequence included in the pseudo-reception signal sequence by using the above-described method. The synchronization deviation is a time difference between a generation timing of the optical pulse and a start timing of a reception period in the probe. The correction unit 64 corrects the pseudo-reception signal sequence based on the synchronization deviation. Specifically, the photoacoustic wave signal sequence included in the pseudo-reception signal sequence is shifted in the depth direction such that the synchronization deviation is apparently eliminated. This shift corresponds to the elimination of the positional deviation caused by the synchronization deviation.

The transformer 66 applies the phase addition to the corrected pseudo-reception signal sequence. The phase addition may be a Fourier transform (Fourier phasing). The corrected and phase-added pseudo-reception signal sequence is output from the transformer 66. The pseudo-reception signal sequence is corrected reception frame data and is corrected reception information.

The PA image generation unit 42 generates a PA image based on the corrected reception frame data. The US image generation unit 40 generates a US image as a tomographic image based on the reception frame data output from the data processing unit 38. The display processing unit 44 composites the PA image with the US image to generate a composite image. The composite image is displayed on the display 46.

In the second embodiment, the reception frame data output from the data processing unit 38 may be given to the PA image generation unit 42 as it is, and the PA image before correction output from the PA image generation unit 42 may be given to the processor 59 (see reference numeral 68). In that case, the corrected PA image is generated by the processing of the processor 59. The corrected PA image corresponds to the corrected reception information.

In the second embodiment, the CPU may function as the processor 59. According to the second embodiment, an advantage of being able to use the reception unit 26C having a general configuration as it is can be obtained.

As described above, with the ultrasonic imaging system according to the embodiment, even in a case where there is a synchronization deviation between the optical pulse period and the reception period, that is, even in a case where there is a time difference between the optical pulse generation timing and the start timing of the reception period, it is possible to display the sound source at the correct position.

What is claimed is:

1. An ultrasonic imaging system comprising:
a light source configured to generate an optical pulse;
an insertion member that is inserted into a living body and that includes an optical absorption element which converts the optical pulse into a photoacoustic wave;
a probe including a plurality of transducers that receive the photoacoustic wave;
an analyzer configured to calculate a synchronization deviation between an optical pulse period in the light source and a reception period in the probe based on a reception signal sequence consisting of a plurality of reception signals that are output in parallel from the plurality of transducers and a pseudo-reception signal sequence corresponding to the reception signal sequence;
a processor configured to apply correction for eliminating a positional deviation caused by the synchronization deviation and phase addition for forming a reception beam to the reception signal sequence or the pseudo-reception signal sequence to output corrected reception information; and
a generator configured to generate a photoacoustic image representing a position of the optical absorption element in the living body based on the corrected reception information.

2. The ultrasonic imaging system according to claim 1,
wherein the synchronization deviation is a time difference between a generation timing of the optical pulse and a start timing of the reception period in the probe.

3. The ultrasonic imaging system according to claim 1,
wherein the processor includes
a correction unit configured to shift a photoacoustic wave signal sequence included in the reception signal sequence or the pseudo-reception signal sequence in a depth direction based on the synchronization deviation, and
a phase addition unit configured to apply the phase addition to the corrected reception signal sequence or corrected the pseudo-reception signal sequence output from the correction unit to generate the corrected reception information.

4. The ultrasonic imaging system according to claim 1,
wherein the processor is configured to apply the correction and the phase addition to the reception signal sequence or the pseudo-reception signal sequence at the same time.

5. The ultrasonic imaging system according to claim 1, further comprising:
a restoration processor configured to apply a restoration processing to reception information generated from the reception signal sequence to generate the pseudo-reception signal sequence,
wherein the analyzer calculates the synchronization deviation based on the pseudo-reception signal sequence.

6. The ultrasonic imaging system according to claim 1,
wherein the analyzer calculates the synchronization deviation based on a photoacoustic wave signal sequence included in the reception signal sequence or the pseudo-reception signal sequence.

7. The ultrasonic imaging system according to claim 6,
wherein the photoacoustic wave signal sequence is composed of a plurality of photoacoustic wave signals corresponding to the plurality of transducers, and
the analyzer calculates the synchronization deviation based on a plurality of propagation times corresponding to the plurality of photoacoustic wave signals.

8. A photoacoustic image generation method comprising:
receiving a photoacoustic wave with a plurality of transducers in a state in which an insertion member including an optical absorption element that converts an optical pulse into the photoacoustic wave is inserted into a living body;
calculating a synchronization deviation between an optical pulse period and a reception period based on a reception signal sequence consisting of a plurality of reception signals that are output in parallel from the plurality of transducers and a pseudo-reception signal sequence corresponding to the reception signal sequence;
applying correction for eliminating a positional deviation caused by the synchronization deviation and phase addition for forming a reception beam to the reception signal sequence or the pseudo-reception signal sequence to generate corrected reception information; and
generating a photoacoustic image representing a position of the optical absorption element in the living body based on the corrected reception information.
